(19) United States
(12) Patent Application Publication
(10) Pub. No.: US 2007/0167772 A1
(43) Pub. Date: Jul. 19, 2007

(54) Apparatus and method for optimized search for displacement estimation in elasticity imaging

(75) Inventor: Emil G. Radulescu, New Haven, CT (US)

Correspondence Address: BACHMAN & LAPOINTE, P.C., 900 CHAPEL STREET, SUITE 1201, NEW HAVEN, CT, 06510, US

(73) Assignee: Aloka Co., Ltd.

(21) Appl. No.: 11/636,215

(22) Filed: Dec. 8, 2006

Related U.S. Application Data
(60) Provisional application No. 60/748,893, filed on Dec. 9, 2005.

(51) Int. Cl. A61B 8/00

(52) U.S. Cl. 600/438

(57) ABSTRACT

A displacement estimation method is described that limits the exhaustive search for all points in a region of interest of a biological tissue by delivering the axial and lateral displacement maps in two phases. During the first phase, the method executes a limited search to determine axial and lateral displacement estimates for a plurality of locations on at least one axial reference line positioned in the ROI. Non-zero estimates form transition points along the axial reference line where one non-zero transition point value differs from another. During the second phase, the method laterally tracks each transition point throughout the ROI using block-matching algorithms or correlation methods. The displacement estimations identify a trajectory of the transition point through the ROI and form a displacement map. The plurality of transition point displacement maps are assembled as a complete displacement map. The resultant displacement map is used to form a tissue strain display.

### REFERENCE TO RELATED APPLICATION

This application claims the benefit of U.S. Provisional Application No. 60/748,893, filed on Dec. 9, 2005, which is incorporated herein by reference in its entirety.

### BACKGROUND OF THE INVENTION

The invention relates generally to the field of elasticity imaging. More specifically, embodiments of the invention relate to methods and systems that efficiently compare data from two ultrasound radio frequency (RF) data frames and derive a tissue displacement map.

Pathological conditions often produce changes in biological tissue stiffness. For example, the tissues of tumors exhibit different mechanical properties than their surrounding tissue as demonstrated by using palpation as a diagnostic tool. Breast and prostate tumors are especially susceptible to changes in mechanical properties.

Many cancers, such as scirrhous carcinoma of the breast, appear as extremely hard nodules. However, a lesion may or may not possess echogenic properties that would make it detectable using conventional ultrasound imaging systems. Prostate or breast tumors may be difficult to distinguish using conventional ultrasound techniques, yet may still be much stiffer than the surrounding tissue.

Recently, experimental elastic modulus data taken for normal and abnormal breast tissues obtained at different ultrasound frequencies and precompression strain levels showed that the differences between the elastic moduli of the different tissues of the breast may be useful in developing methods to distinguish between benign and malignant tumors. Tissues of the prostate were also examined as cancers of the prostate are also significantly stiffer than normal tissue. Similar data indicating differences between the elastic moduli for normal and abnormal prostate tissues were also reported.

The imaging modality that facilitates the display of mechanical properties of biological tissue is called elastography. Elastography is an emerging method in which stiffness or strain images of soft tissue are used to detect tumors. When a mechanical compression is applied, the tumor deforms less than the surrounding tissue, i.e., the strain in the tumor is less than the surrounding tissue.

The purpose of elastography is to display an image of the distribution of a physical parameter related to the mechanical properties of the tissue for clinical applications. Elasticity imaging consists of inducing an external or internal motion to the suspect tissue and evaluating the response of the tissue using conventional diagnostic ultrasound imaging and correlation techniques.

Each elasticity imaging application comprises three functional components. First, the data is captured during an externally or internally applied tissue motion or deformation. Second, the tissue response is evaluated by determining displacement, stress and strain. Lastly, the elastic modulus of the tissue is reconstructed using the theory of elasticity. The last step involves implementing the theory of elasticity into modeling and solving the inverse problem from strain and boundary conditions to a modulus of elasticity. Since modeling elasticity depends on the structure of the biological tissue and boundary conditions, implementation of the last function is cumbersome and typically not performed for commercial applications. The evaluation and display of tissue strain in the second function is considered to deliver an accurate reproduction of the tissue's mechanical properties.

The most frequently used modality is static elasticity imaging. In this application, a small quasi-static compressive force is applied to the tissue using the ultrasound imaging transducer. The force can be applied either using motorized compression fixtures or using freehand scanning. The radio frequency (RF) data acquired prior to and during compression is recorded and compared to estimate the local axial and lateral motions using correlation methods. The estimated motions along the ultrasound propagation direction represent the axial displacement map of the tissue and are used to determine an axial strain map. The strain map is then displayed as a gray scale or color-coded image and is called an elastogram.

While the majority of elasticity image processing has been performed off-line, real-time elasticity imaging applications for use in clinical environments is a primary concern. Real-time elasticity imaging is needed to process the ultrasonic image data such that patient scanning time is minimal and diagnostically relevant elasticity images are immediately produced. Real-time elasticity imaging systems are capable of displaying ultrasonic B-mode images and strain images on the same user display. The combined display aids in assessing the clinical relevance of the derived strain images.

Real-time processing of ultrasonic image data allows for freehand compression and scanning of a suspect area rather than needing a slow and bulky motorized compression fixture. Freehand compression, as opposed to motorized compression, allows for a manageable and user-friendly scanning process for use in a larger variety of scanning locations. Its disadvantage, however, consist of exhaustive operator training, as the sonographer constantly needs to adjust the compression technique to obtain strain. images of good quality. To obtain consistent strain images exhibiting superior elasticity dynamic range DRₑ, and signal-to-noise ratio SNRₑ, the sonographer needs to maintain a constant compression rate while avoiding lateral and out-of-plane tissue motions. Moreover, the compression has to be performed exclusively on the axial direction of the imaging transducer while maintaining a certain speed and repetition period.

Given the advantages of real-time ultrasonic echo data processing, there is a need for an efficient method to produce accurate and reliable elasticity images. The most time-intensive aspect of processing RF data is the estimation of motions along the ultrasound propagation direction. There is therefore a need to optimize this process.

Conventional tissue displacement algorithms determine the axial and lateral displacement maps by employing block-matching algorithms or by correlation means, systematically carrying out search procedures for each point from a given region of interest (ROI). However, in determining the axial and lateral displacement maps by employing block-matching algorithms or by correlation means, the methods do not optimize the amount of search procedures conducted. As a result, the apparatus requires a considerable amount of time to generate a display of tissue strain.

Consequently, there exists a need for a computational efficient algorithm that optimally reduces the amount of time necessary to complete the displacement estimation for elasticity imaging and generate a display of tissue strain by an imaging apparatus.

### SUMMARY OF THE INVENTION

Although there are various methods and systems that process ultrasound RF and data into a tissue displacement map, such methods and systems are not completely satisfactory. The inventor has discovered that it would be desirable to have methods and systems that efficiently process ultrasound image data into a tissue displacement map for real-time diagnostic imaging applications.

The method of the invention limits the exhaustive search for all points in an ROI by delivering the axial and lateral displacement maps in two phases.

During the first phase, the method executes a limited search to determine axial and lateral displacement estimates for a plurality of locations on at least one axial reference line positioned in the ROI. The estimates are at an elasticity imaging resolution determined by an operator. Non-zero displacement estimates that are returned may be multiples of fixed, predefined increments. The non-zero increments in estimates form transition points along the axial reference line where one non-zero transition point value differs from another.

During the second phase, the method laterally tracks each transition point throughout the ROI using block-matching algorithms or correlation methods. The displacement estimations identify a trajectory of the transition point through the ROI and form a displacement map. The plurality of transition point displacement maps are assembled as a complete displacement map. The resultant displacement map is used to form a tissue strain display.

One aspect of the invention provides methods for determining a displacement map between first and second data frames containing RF data values. Methods according to this aspect of the invention preferably start with indexing the RF data values for the first and second frames with a sample resolution and a display resolution, creating at least one axial reference line of RF data values having a plurality of positions indexed at the display resolution in the first RF frame, using a block-matching algorithm with reference blocks centered on the axial reference line positions, determining the best axial displacement estimations for the axial reference line positions in the second RF frame, storing the axial displacement estimation values for each axial reference line position, and defining an axial reference line position as a transition point, wherein when adjacent axial reference line positions have different values, the axial reference line position having the greater value is a transition point.

Another aspect of the method includes performing a displacement estimation at a lateral location adjacent to a transition point, performing lateral tracking comprising if the displacement estimation for the adjacent lateral location equals the transition point value, performing additional displacement estimations for adjacent axial locations until a displacement estimation for an adjacent axial location is less than the transition point value, if the displacement estimation for the adjacent axial location is greater than the transition point value, performing additional displacement estimations for adjacent axial locations until a displacement estimation for an adjacent axial location is less than the transition point value, if the displacement estimation for the adjacent axial location is less than the transition point value, performing additional displacement estimations for adjacent opposite axial locations until a displacement estimation for an adjacent axial location is the same as the transition point value, determining the axial location displacement estimation value that is the same as the transition point value prior to the axial location that is less than the transition point value is a trajectory point, using the determined trajectory point, repeating lateral tracking to determine a next trajectory point until there are no more lateral locations to consider, and assembling a displacement map corresponding to a transition point from the plurality of corresponding trajectory points.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is an exemplary search pattern for a block-matching algorithm.

FIG. 2 is an exemplary search pattern for the method of the invention.

FIG. 3 is a block diagram of an exemplary transition point determination method.

FIG. 4 is a block diagram of an exemplary lateral tracking method.

FIG. 5 is an exemplary plot of displacement estimation versus axial depth of transition point positions for one representative axial reference line where displacements are estimated from.

FIG. 6 is an exemplary diagram of laterally tracking one transition point where one axial reference line is employed.

FIG. 7 is an exemplary diagram of laterally tracking one transition point where two axial reference lines are employed.

FIG. 8 is an exemplary diagram of laterally tracking three different transition points where three axial reference lines are employed.

FIG. 9 is an exemplary framework of the modules of the invention.

### DETAILED DESCRIPTION

Embodiments of the invention will be described with reference to the accompanying drawing figures wherein like numbers represent like elements throughout. Further, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. The terms “mounted,” “connected,” and “coupled” are used broadly and encompass both direct and indirect mounting, connecting, and coupling. Further, “connected” and “coupled” are not restricted to physical or mechanical connections or couplings.

The invention is not limited to any particular software language described or implied in the figures. A variety of alternative software languages may be used for implementation of the invention. Some components and items are illustrated and described as if they were hardware elements, as is common practice within the art. However, various components in the method and system may be implemented in software or hardware.

Embodiments of the invention provide methods, systems, and a computer-usable medium storing computer-readable instructions that efficiently process ultrasound RF image data into a tissue displacement map for real-time diagnostic imaging applications. The invention efficiently compares data from two ultrasound radio frequency (RF) data frames and derives a tissue displacement map. The invention is a modular framework and may be deployed as hardware resident in an enclosure having an onboard power supply, or as software as an application program tangibly embodied on a program storage device for executing with a computer or processor. The application code for execution may reside on a plurality of different types of computer readable media.

By way of background, ultrasonography (sonography) uses a probe containing a plurality of acoustic transducers to send pulses of sound into a material. A sound wave is typically produced by creating short, strong pulses of sound from a phased array of piezoelectric transducers encased in a probe. The frequencies used for medical imaging are generally in the range of from 1 to 13 MHz which are medium to high radio frequencies (RF) and produce a single, focused arc-shaped sound wave from the sum of all the individual pulses emitted by the transducer. Higher frequencies have a correspondingly lower wavelength and yield higher resolution images.

Whenever the sound wave encounters a material with a different acoustical impedance, part of the sound wave is reflected, which the probe detects as an echo. The return sound wave vibrates the transducer's elements and turns that vibration into electrical pulses that are sent from the probe to a processor where they are processed and transformed into a digital image. The time it takes for the echo to travel back to the probe is measured and used to calculate the depth of the tissue interface causing the echo. The greater the difference between acoustic impedances, the larger the echo is. The difference between gases and solids is so great that most of the acoustic energy is reflected, and so imaging of objects beyond that region is not possible.

The speed of sound is different in different materials, and is dependent on the acoustic impedance of the material. However, an ultrasound scanner assumes that the acoustic velocity is constant at 1540 m/s. Although part of the acoustic energy is lost every time an echo is formed, this effect is small compared to the attenuation of sound due to absorption.

To generate a 2-dimensional image, the ultrasound beam is swept, either mechanically, or electronically using a phased array of acoustic transducers. The received RF data is further processed and used to construct the conventional ultrasound image.

The processor must determine from each received echo, which transducer elements received the echo since there are multiple elements on a transducer, the strength of each echo, and the time difference from when the sound was transmitted and when the echo was received. Once a determination is made, the processor can locate which value in a frame is present and to what magnitude.

The received data is referred to as RF data values and its representation is similar to that of a matrix. For example, with I indentifying rows (axial) and J identifying columns (lateral) where I=1, 2, 3, . . . , M and J=1, 2, 3, . . . , N. The RF data values a_{I,J}, are typically bipolar (±) multi-bit values. For example, a 2048×128 RF data frame may have 262, 144 values a_{I,J}.

For elasticity imaging, the invention processes ultrasound RF data in real-time using intelligent search strategies in conjunction with block-matching methods. The invention maximizes throughput by minimizing computation resources.

The invention provides displacement estimates of tissue motions between two RF data frames in axial (I) and lateral (J) directions. A first RF frame may be a non-compressed tissue section, the second RF frame is of the same ultrasound tissue section, but compressed in an axial (I) direction with regard to tissue surface.

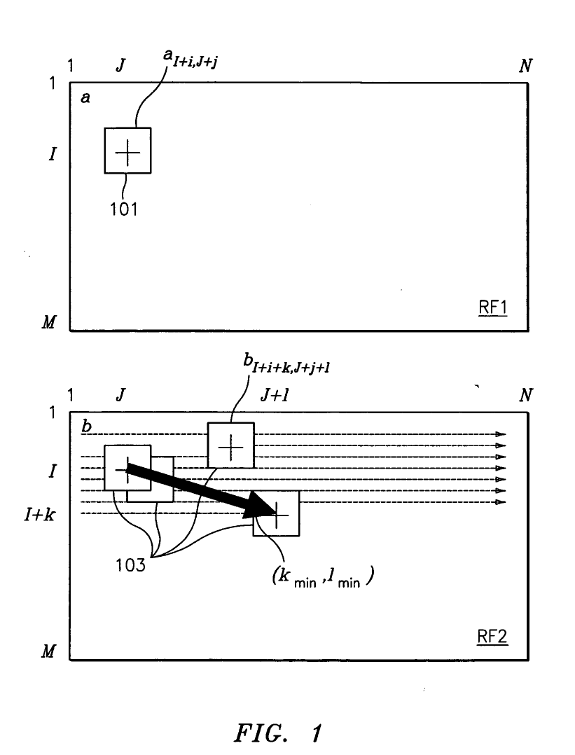

Shown in FIG. 1 is an exemplary conventional search pattern for a block-matching algorithm. An array, or block of RF data values a_{I+i,J+j} from the first RF frame RF1 is selected as a kernel 101 surrounding the center location. i and j are local vertical and horizontal indices, respectively. The kernel is compared to blocks 103 of the same size b_{I+i+k,J+j+l} from RF data frame RF2 corresponding to the same tissue section, but compressed in the axial (I) direction. The difference between the location found in the second frame RF2 for the candidate block 103 exhibiting the best match I+k,J+l, and the location of the reference block 101 in the first frame RF1 I,J is the displacement k,l, and forms a vector for that respective reference block 101 indicating motion in both axial (I) and lateral (j) directions. The best match is shown as a bold arrow. A displacement map is collated from a plurality of different reference block/best candidate block matches showing axial and lateral displacements between the first RF1 and second RF2 RF frames.

The reference block 101 is indexed, from left to right, top to bottom, across the entire second data frame RF2 as shown in FIG. 1, or within a predetermined search area or ROI, as the reference block 101 is compared with candidate blocks 103 in the second RF frame RF2. The indexing of candidate block 103 locations to search may be performed from one data position b_{I+i+k,J+j+l} to the next b_{I+i+k,J+j+(l+1)} or b_{I+i+(k+1),J+j+l}. For each new reference block/candidate block comparison, a fitness score is calculated. A variety of methods exist to determine similarity, or fitness, between a reference block 101 and a candidate block 103.

Block-matching algorithms and other comparison techniques may be employed as described above to determine axial and lateral displacement maps of an ROI at a given display resolution. The axial and lateral display resolution may be determined by the number of blocks 101 per the axial dimension of the ROI and the lateral dimension of the ROI, respectively. A displacement estimation becomes extremely time consuming as the block-matching algorithm needs to be calculated for every block 101 to yield displacement values.

The displacement estimation method of the invention limits the amount of searches for deriving displacement maps of the RF data in the ROI. The method employs a conventional block-matching procedure to perform a limited search to determine axial and lateral displacement estimates for a plurality of locations on at least one axial reference line positioned in the ROI. The resolution in the axial direction may be the display resolution.

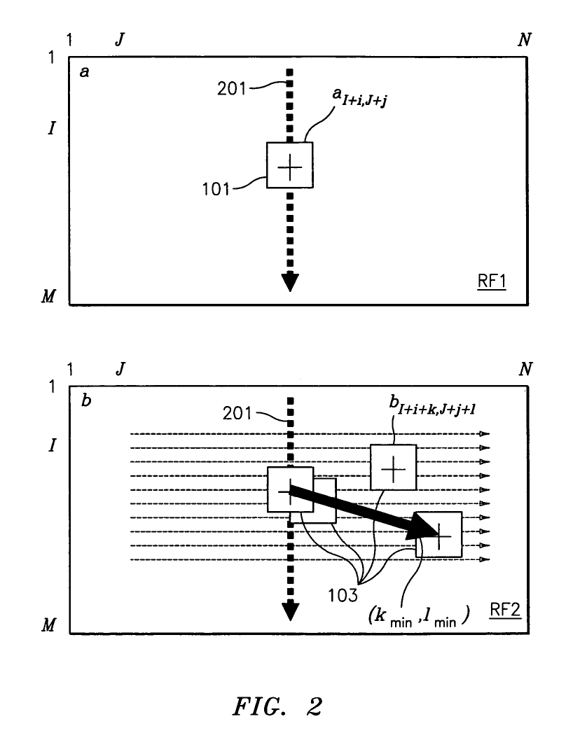

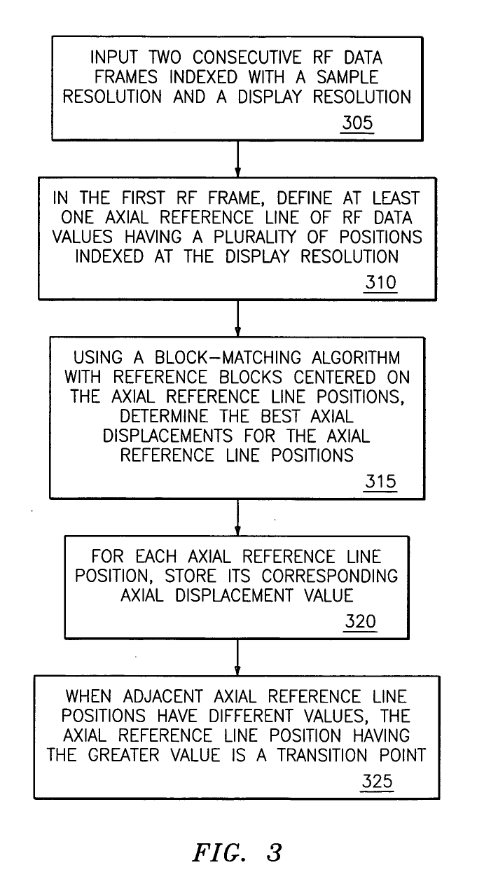

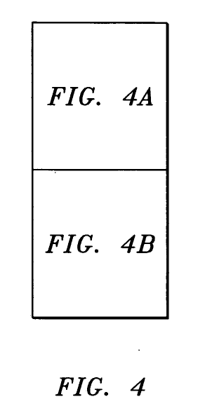

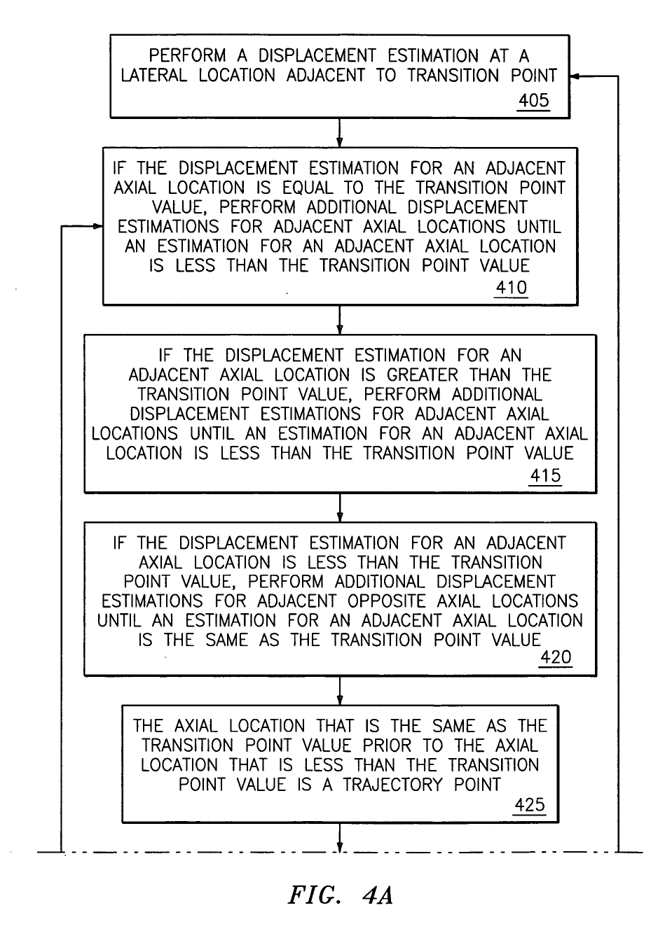

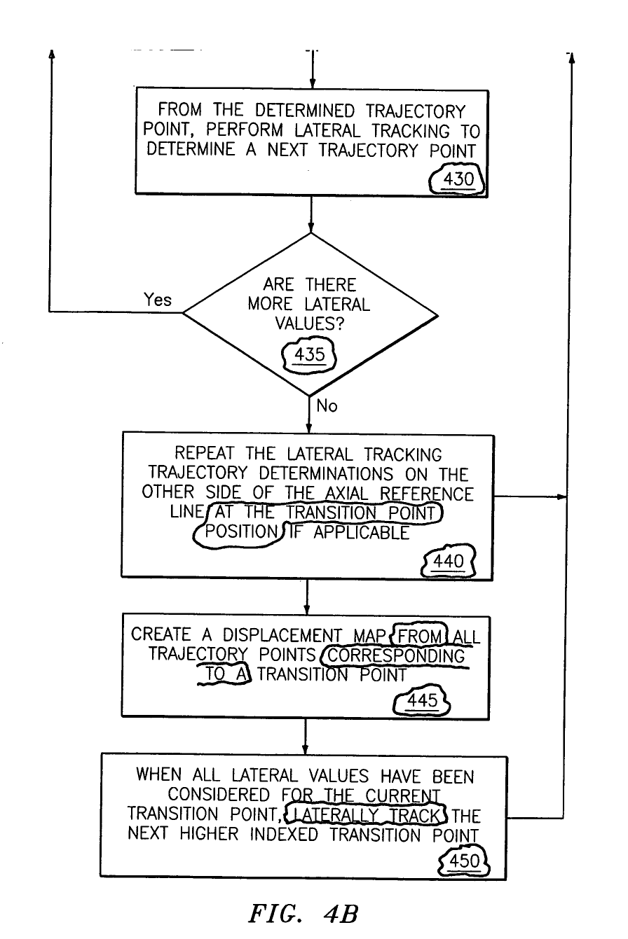

FIG. 2 shows one axial reference line 201 within an ROI. FIGS. 3 and 4 show the method. The method may employ more than one axial reference line 201, preferably three. The position of the axial reference lines may be equally spaced within the ROI. For example, if one line is considered, it may be positioned in the middle of the ROI (as in FIG. 2). If three lines are considered, the axial reference lines may divide the ROI into thirds. Unlike prior art methods where search procedures are carried out for every RF data value within the ROI, the invention economizes and optimizes the search procedure by using at least one axial reference line that reduces the number of calculations used to find displacement estimates. To aide in teaching the method of the invention, one axial reference line 201 will be taught.

FIG. 2 shows one axial reference line 201 centered in a first RF data frame RF1. The method uses a block-matching algorithm that determines the axial and lateral displacements of a plurality of reference block center locations positioned on the axial reference line 201 (steps 305, 310). A plurality of locations are indexed along the length of the axial reference line 201 typically at the display resolution. For example, the sample resolution of an axial reference line may be 1000 samples, whereas the display resolution may be every 100 samples (100, 200, 300, 400, . . . ) yielding 10 axial reference line 201 positions. The positions represent the center locations of reference blocks 101 used in the block-matching algorithm. The resultant axial display resolution of the ROI is determined by the number of axial reference line positions.

For each axial reference line 201 position, a block of RF data values a_{I+i, J+j} from the first RF frame RF1 is selected as a kernel 101 surrounding the center location. i and j are local vertical (axial) and horizontal (lateral) indices, respectively. The kernel is compared to blocks 103 of the same size b_{I+i+k,J+j+l} from RF data frame RF2 corresponding to the same tissue section, but compressed in the axial I direction. The difference between the location found in the second frame RF2 for the candidate block 103 exhibiting the best match I+k,J+l, and the location of the reference block 101 in the first frame RF1 I,J is the displacement k, l and forms a vector for that respective reference block 101 indicating motion in both axial (I) and lateral (j) directions. The best match is shown as a bold arrow (step 315) and corresponds to the lag kₘᵢₙ,lₘᵢₙ. As the reference block 101 for comparison follows the axial reference line 201, the horizontal index (J) is a constant.

$$J = \frac{N}{2} .$$

The blocks or kernels 101 may have dimensions such that when the candidate blocks are placed on the axial reference line 201 positions, the borders of the kernels may meet. Alternatively, the borders may have dimensions such that the borders overlap, or may have dimensions where the borders do not touch. The axial displacements versus depth for each axial reference line 201 position are collated from a plurality of different reference block/best candidate block matches showing axial and lateral displacements between the first RF1 and second RF2 RF data frames.

As a reference block 101 is compared with candidate blocks 103 in the second RF frame RF2. The indexing of candidate block 103 locations to search may be performed from one data position b_{I+i+k,J+j+l} to the next b_{I+i+k,J+j+(l+1)} or b_{I+i+(k+1), J+j+l}. For each new reference block/candidate block comparison, a fitness score is calculated. A variety of methods exist to determine similarity, or fitness, between a reference block 101 and a candidate block 103.

The results of the search using the axial reference line 201 positions as beginning coordinates, are axial displacements matching the number of axial reference line 201 positions (step 320). The axial displacements may be quantified in RF data value samples that correspond to the sample frequency of the beamformed RF signal. The axial lag kₘᵢₙ is an integer value indicating the axial displacement in RF samples quanta for one candidate block 101 starting at an axial reference line 201 position.

After the search is performed for each axial reference line 201 position, non-zero increments at the sample resolution are stored for each axial reference line 201 position. When adjacent axial reference line 201 positions have different values, the axial reference line 201 position having the greater value is referred to as a transition point (step 325).

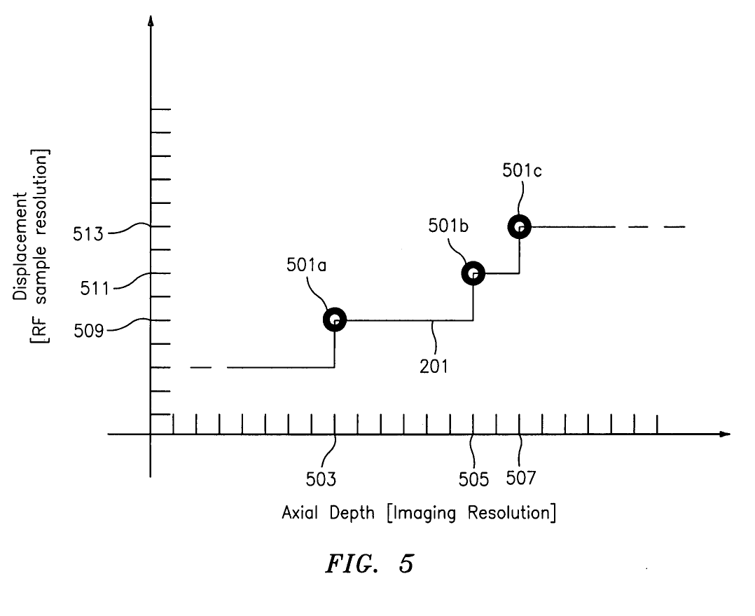

An axial displacement estimation versus axial depth for a representative axial reference line 201 is shown in FIG. 5. The units of axial depth are at the image resolution. The units of displacement are RF sample quanta. The axial displacements for each axial reference line 201 position at the image resolution are analyzed, and wherever the displacement estimates exhibit an increase or decrease, of one or multiples of fixed predefined increments from the estimated values of adjacent positions, the positions having the greater value are flagged as transition points 501. The plot shows three transition points 501a, 501b, 501c each located at a different axial reference line 201 position 503, 505, 507, each having a different transition point value (displacement) 509, 511, 513.

An increase, or positive step between displacement estimates from two adjacent axial reference line 201 positions may be observed. As a sign convention, positive displacements (step-up) are associated with axial tissue compression from one RF frame to another. Negative displacements (step-down) are associated with axial tissue decompression, or relaxation, from one RF frame to another.

If the block-matching algorithm employed delivers an axial lag kₘᵢₙ with sub-RF sample resolution, each position's axial displacement may be algebraically scaled and rounded to be expressed in a discrete, quantified estimated value.

Each transition point position is a starting location to perform lateral tracking of the transition point throughout the ROI. For each transition point, where the magnitude of each transition may be a multiple of a fixed predefined increment, may be tracked (identified) through all lateral locations across the ROI. The lateral progression for identifying transition points corresponding to each axial reference line 201 position where transition points occur may be performed by employing a block-matching algorithm or other correlation method at the axial and lateral display resolution, using the previously determined axial reference line 201 transition points.

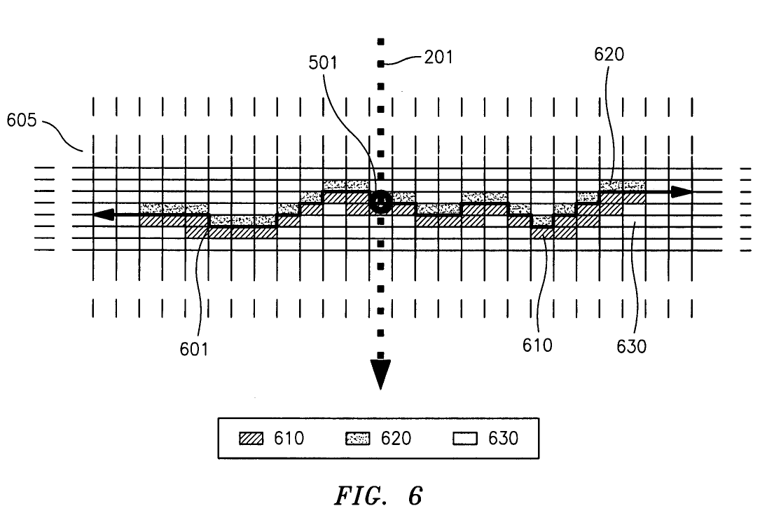

FIG. 6 shows the lateral tracking method for one transition point 501 across an ROI. The axial and lateral display resolution is depicted as a grid 605. Since the axial display resolution may not be at the same resolution as the lateral display resolution, the grid 605 is depicted using unequal divisions on the axial direction versus the lateral direction.

The lateral track 601 corresponding to the transition point 501 may be built on either side of the axial reference line 201, as indicated by the end arrows. The axial displacement value associated with the transition point 501 is tracked laterally throughout the ROI. A block-matching algorithm may be employed to perform displacement estimations at positions of the reference block 101 situated laterally on either side of a transition point 501 (step 405). The results of the displacement estimations are indicated with dashed rectangles 610 for axial displacements equal to the transition point 501 value. Displacement estimations smaller than the transition point 501 value are indicated with solid gray rectangles 620.

Displacement estimations may be performed for axial positions above or below the transition point lateral position (steps 410, 415, 420). Typically, 2 to 3 positions above or below are required. The displacement estimations are used to indicate when a transition occurs from the transition point value 501 to a displacement estimate having a lower value. The axial location that is the same as the transition point value adjacent to an axial location that is less than the transition point value is referred to as a trajectory point (step 425).

From one determined trajectory point, a next trajectory point is determined (step 430). The method repeats the above steps (steps 410, 415, 420, 425), moving away from the axial reference line 201 until there are no more lateral values to consider (step 435). If the axial reference line 201 divides the ROI, trajectory determinations are performed for the other side of the reference line 201 (step 440) at the transition point 501 position. Once all lateral values have been considered, the method assembles a profile, or displacement map, from all of the trajectory points corresponding to a respective transition point (step 445). The lateral tracking method is repeated for a next, higher indexed transition point (step 450) until all transition points have been considered.

Finally, each trajectory corresponding to a transition point may be assembled into one displacement map for the ROI. Any remaining locations between transition point trajectory maps that have not been associated with a transition point 630, or that have not had a displacement estimation performed, may be assigned a constant values. If a remaining location is between transition point displacement map boundaries having the same transition point value, the remaining location may be assigned the same value as the bordering transition point values. If a remaining location is between borders of transition point displacement maps having different transition point values, the remaining location may be assigned the value of the transition point having a lesser value. The areas between transition point displacement map boundaries may be considered plateaus between the trajectories corresponding to respective transition points.

It may be seen from the combination of the vertical and lateral tracking methods that an economy of operation has been achieved since the block-matching algorithm has not been used to determine every location of the displacement map. The display locations shown by empty rectangles 630 in FIG. 6 indicate locations where displacement estimations were not necessary, and may be assigned a corresponding transition point value (as described above) thereby decreasing the computation time necessary to deliver the entire displacement map. It may also be seen that when performing lateral tracking, the displacement estimation values calculated using block-matching that correspond to each adjacent axial determination may be stored and reexamined if a later transition point trajectory path is close by. Rather than having to perform a block-matching calculation for the same block when performing a later transition point trajectory, the method reuses a previously calculated displacement estimation to determine a later trajectory.

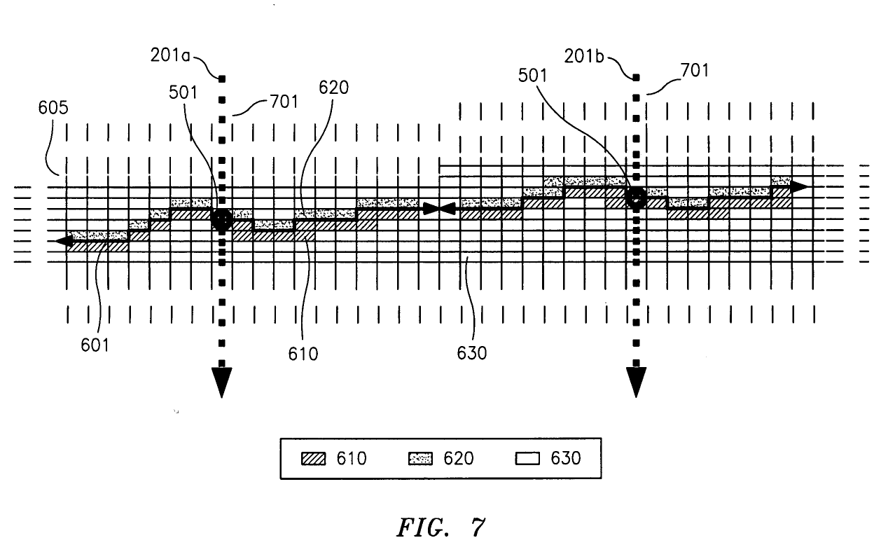

Shown in FIG. 7 is an application where more than one axial reference line 201 is used. The above described method is applicable for each axial reference line 201a, 201b. By using multiple axial reference lines 201a, 201b parallel processing may be employed, during the axial reference line position phase and the lateral tracking phase. While the axial reference line 201a, 201b positions 701 will be the same, each axial reference line 201a, 201b may have the same transition points 501, but located at a different axial positions. The trajectories originating from like transition points 501 on each axial reference line 201a, 201b starting from either side will converge into one, completing a displacement map 601 for that respective transition point 501. Trajectories corresponding to the same transition point are characterized by an identical axial displacement value.

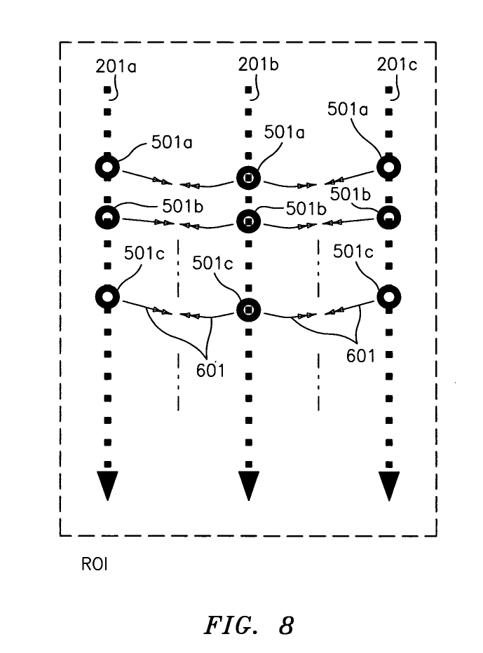

More generally, FIG. 8 shows an application of the invention employing three axial reference lines 201a, 201b, 201c with multiple transition points 501a, 501b, 501c and the corresponding trajectories 601.

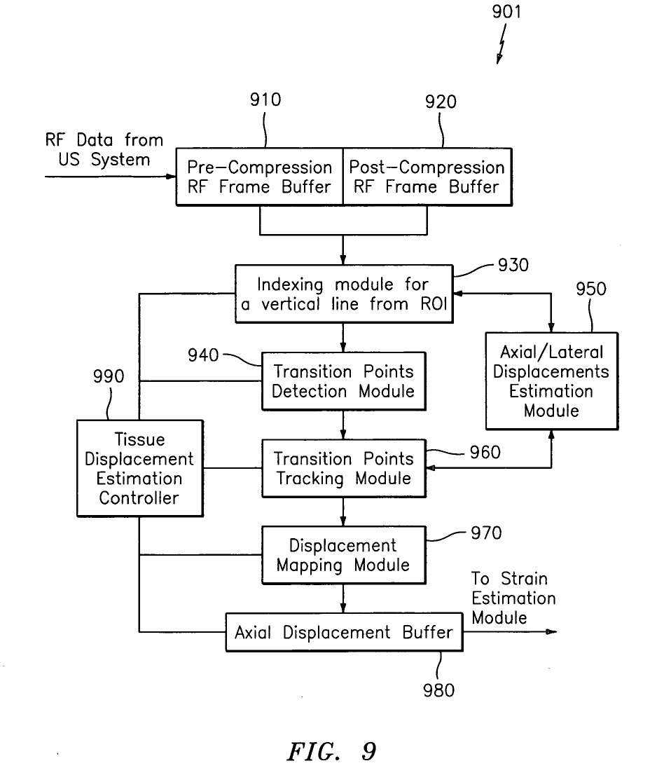

Shown in FIG. 9 is a corresponding framework 901 of the various modules that comprise the invention. The invention is a modular framework and may be deployed as software, as hardware, or as a combination of software and hardware.

The invention framework 901 receives RF frame data from an ultrasound system. The coupled modules include buffers for pre-compression 910 and post-compression 920 RF frame data, an indexing module for a vertical line from ROI 930, an axial/lateral displacements estimation module 950, a transition points detection module 940, a transition points tracking module 960 and a displacement mapping module 970. A controller 990 provides for synchronization and communication between the activities of the modules mentioned above and the resulted axial displacements are stored in an axial displacement buffer 980. From the axial displacement buffer 980, the axial displacements are sent to the strain estimation module of the elasticity imaging system.

RF data values corresponding to two RF frames RF1, RF2 are output from an ultrasound system and are input to the framework 901 for processing. The two RF data frames are stored in corresponding pre-compression 910 and post-compression 920 RF frame buffers. The pre-compression data is collected prior to tissue compression and the second RF data frame is collected after tissue compression.

The indexing module for an axial reference line from an ROI 930 reads the RF data from the two input buffers 910, 920, allocates memory and assigns addresses of where to store and read the RF data values during processing. After memory allocation, the axial/lateral displacements estimation module 950 determines the axial and lateral displacements of an axial reference line.

The transition points detection module 940 analyzes the axial displacements obtained and determines the transition points, which are tracked laterally by the transition points tracking module 960, using the axial/lateral displacements estimation module 950. The tracked transition points are mapped by the displacement mapping module 970 and the remaining locations that have not been associated with a transition point are assigned constant values equal to the estimated displacement of at least one axial transition point of closest proximity. The results, stored in axial displacement buffer 980 are then passed to the strain estimation module.

One or more embodiments of the present invention have been described. Nevertheless, it will be understood that various modifications may be made without departing from the spirit and scope of the invention. Accordingly, other embodiments are within the scope of the following claims.

What is claimed is:

1. A method for determining a displacement map between first and second data frames containing RF data values comprising:
indexing the RF data values for the first and second frames with a sample resolution and a display resolution;
creating at least one axial reference line of RF data values having a plurality of positions indexed at the display resolution in the first RF frame;
using a block-matching algorithm with reference blocks centered on the axial reference line positions, determining the best axial displacement estimations for the axial reference line positions in the second RF frame;
storing the axial displacement estimation values for each axial reference line position; and
defining an axial reference line position as a transition point, wherein when adjacent axial reference line positions have different values, the axial reference line position having the greater value is a transition point.

2. The method according to claim 1 wherein display resolution is a multiple of sampling resolution.

3. The method according to claim 2 wherein a displacement estimation is obtained using a block-matching algorithm between a reference block located in the first RF data frame and a plurality of candidate blocks located in the second RF data frame.

4. The method according to claim 3 further comprising:
performing a displacement estimation at a lateral location adjacent to a transition point;
performing lateral tracking comprising:
if the displacement estimation for the adjacent lateral location equals the transition point value, performing additional displacement estimations for adjacent axial locations until a displacement estimation for an adjacent axial location is less than the transition point value;
if the displacement estimation for the adjacent axial location is greater than the transition point value, performing additional displacement estimations for adjacent axial locations until a displacement estimation for an adjacent axial location is less than the transition point value;
if the displacement estimation for the adjacent axial location is less than the transition point value, performing additional displacement estimations for adjacent opposite axial locations until a displacement estimation for an adjacent axial location is the same as the transition point value;
determining the axial location displacement estimation value that is the same as the transition point value prior to the axial location that is less than the transition point value is a trajectory point;
using the determined trajectory point, repeating lateral tracking to determine a next trajectory point until there are no more lateral locations to consider; and
assembling a displacement map corresponding to a transition point from the plurality of corresponding trajectory points.

5. The method according to claim 4 further comprising, proceeding to a next higher indexed transition point.

6. The method according to claim 5 further comprising for each transition point, assembling a corresponding displacement map.

7. The method according to claim 6 further comprising assembling one displacement map from all transition point displacement maps.

8. The method according to claim 7 wherein areas on the one displacement map between two transition point maps are assigned the transition point value of the transition point having the lesser value.

9. A system for determining a displacement map between first and second data frames containing RF data values comprising:
means for indexing the RF data values for the first and second frames with a sample resolution and a display resolution;
means for creating at least one axial reference line of RF data values having a plurality of positions indexed at the display resolution in the first RF frame;
using a block-matching algorithm with reference blocks centered on the axial reference line positions, means for determining the best axial displacement estimations for the axial reference line positions in the second RF frame;
means for storing the axial displacement estimation values for each axial reference line position; and
means for defining an axial reference line position as a transition point, wherein when adjacent axial reference line positions have different values, the axial reference line position having the greater value is a transition point.

10. The system according to claim 9 wherein display resolution is a multiple of sampling resolution.

11. The system according to claim 10 wherein a displacement estimation is obtained using a block-matching algorithm between a reference block located in the first RF data frame and a plurality of candidate blocks located in the second RF data frame.

12. The system according to claim 11 further comprising:
means for performing a displacement estimation at a lateral location adjacent to a transition point;
means for performing lateral tracking comprising:
if the displacement estimation for the adjacent lateral location equals the transition point value, means for performing additional displacement estimations for adjacent axial locations until a displacement estimation for an adjacent axial location is less than the transition point value;
if the displacement estimation for the adjacent axial location is greater than the transition point value, means for performing additional displacement estimations for adjacent axial locations until a displacement estimation for an adjacent axial location is less than the transition point value;
if the displacement estimation for the adjacent axial location is less than the transition point value, means for performing additional displacement estimations for adjacent opposite axial locations until a displacement estimation for an adjacent axial location is the same as the transition point value;
means for determining the axial location displacement estimation value that is the same as the transition point value prior to the axial location that is less than the transition point value is a trajectory point;
using the determined trajectory point, means for repeating lateral tracking to determine a next trajectory point until there are no more lateral locations to consider; and
means for assembling a displacement map corresponding to a transition point from the plurality of corresponding trajectory points.

13. The system according to claim 12 further comprising, means for proceeding to a next higher indexed transition point.

14. The system according to claim 13 further comprising for each transition point, means for assembling a corresponding displacement map.

15. The system according to claim 14 further comprising means for assembling one displacement map from all transition point displacement maps.

16. The system according to claim 15 wherein areas on the one displacement map between two transition point maps are assigned the transition point value of the transition point having the lesser value.

17. A method for performing elasticity imaging between first and second data frames comprising:
estimating displacements between the first and second data frames for positions along at least one axial reference line in a region of interest in the first data frame;
defining transition points for positions on the at least one axial reference line;
tracking the transition points laterally through the region of interest;
assembling displacement maps from trajectories for each transition point;
assembling one displacement map from all transition point displacement maps; and
between transition point displacement maps, assigning the area a value belonging to the transition point having the lesser value.
